(19) United States
(12) Plant Patent Application Publication
(10) Pub. No.: US 2013/0326771 P1
(43) Pub. Date: Dec. 5, 2013

(54) Cherry tree named Sweet Lorenz

(50) ;

(71) Applicant: Alma Mater Studiorum-Universita degli Studi di Bologna, Bologna (IT)

(72) Inventors: Stefano LUGLI, Modena (IT); Riccardo CORREALE, Bologna (IT); Michelangelo GRANDI, Castenaso (BO) (IT)

(21) Appl. No.: 13/986,701

(22) Filed: May 28, 2013

(30) Foreign Application Priority Data
May 25, 2012 (QZ) 2012/1158

(51) Int. Cl. A01H 5/00

(52) U.S. Cl. PLT/181

(57) ABSTRACT

PA2UNIBO ‘Sweet Lorenz’ is a novel cherry tree of the genus/species Prunus avium derived from a seedling of unknown cross which was planted at Vignola, Modena Province, Italy. It was initially propagated by grafting to root stocks of varying vigor and, after testing in districts and plantings of differing types, proved to have the qualities proper to a promising new cultivar for the market.

### CROSS-REFERENCE TO RELATED APPLICATIONS

This application claims priority of pending EU Plant Patent for ‘Sweet Lorenz’, which was filed on 25 May 2012 as Application No. 2012/1158 in the name of Applicant (holder) Alma Mater Studiorum—Università degli Studi di Bologna, the entire contents of which is expressly incorporated herein by reference. EU Trademark for ‘Sweet Lorenz’ was released on 2 Jan. 2013 as Registration No. 011043973 of Applicant (holder) Alma Mater Studiorum—Università degli Studi di Bologna.

### LATIN NAME OF THE GENUS/SPECIES OF THE PLANT CLAIMED (CULTIVAR)

Prunus avium.

### VARIETY DENOMINATION

PA2UNIBO

### FIELD OF THE INVENTION

Prunus avium PA2UNIBO is derived from a seedling of unknown cross and originally called DCA BO A1C27. It was planted at Vignola, Modena Province, Italy, in February 2002 and first cropped in 2004. It was initially propagated by grafting to root stocks of varying vigor, tested in different growing districts and planting densities and proved to have the properties proper to a promising new cultivar for the market. It picks about 10 after ‘Burlat’ (or about 8 days before ‘Bing’) at the same date as Celeste® Sumpaca*.

### SUMMARY OF THE INVENTION

The parentage of the variety is unknown. The name of a comparison variety is ‘Celeste’®Sumpaca*. Compared to ‘Celeste’® Sumpaca*, PA2UNIBO has larger, firmer fruit of more uniform ripening and higher sugar content. PA2UNIBO was selected for its high qualities, including tree growth and yield performance, and fruit appearance and excellent taste-flavor properties. Its distinctive traits include early-mid season picking, uniform maturing and large-sized fruit of firm flesh. Its other most distinctive traits include mid-season, uniform ripening and large, firm-fleshed fruit. The date of discovery/cross was in the year 2000. The location of the discovery was Vignola, Modena Province, Italy. The date of the first asexual propagation was in 2008 in Vignola, Modena Province, Italy. PA2UNIBO was originally propagated on ‘Colt’ and ‘Gisela 6’ root stocks of differing vigor in trial fields of Bologna University to test growth and yield performance under different planting systems

### BRIEF DESCRIPTION OF THE DRAWINGS

The accompanying photographs show typical specimens of the new variety as depicted in color as nearly true as is reasonably possible in color illustrations of this character.

FIG. 1 illustrates the detail of fully-open blossoms of PA2UNIBO.

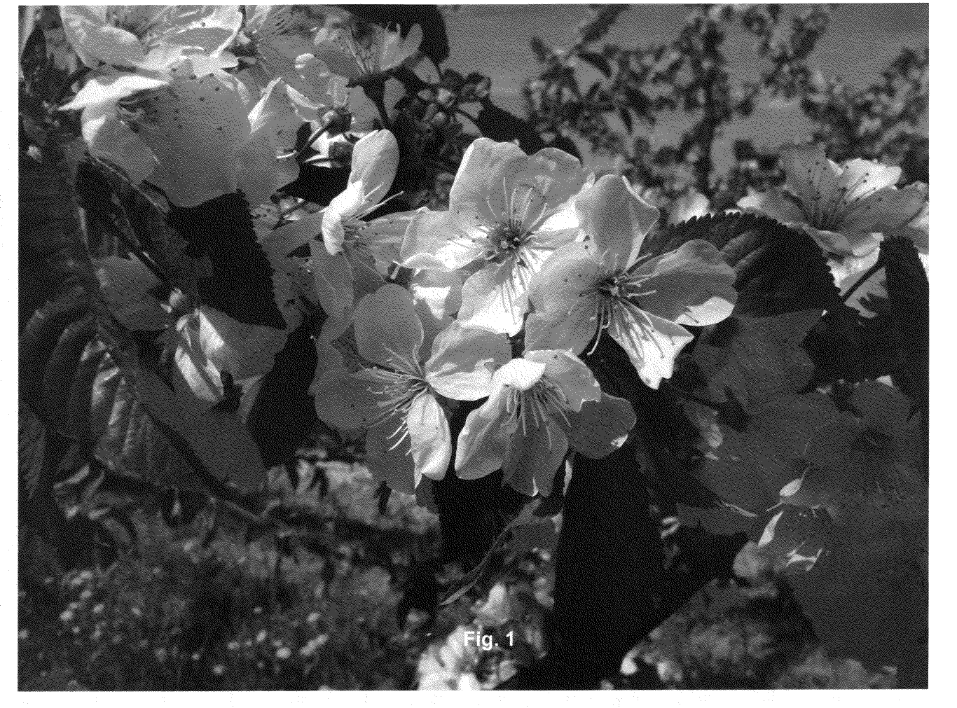

FIG. 2 shows the flower, full front, in profile and with separated petals, of PA2UNIBO.

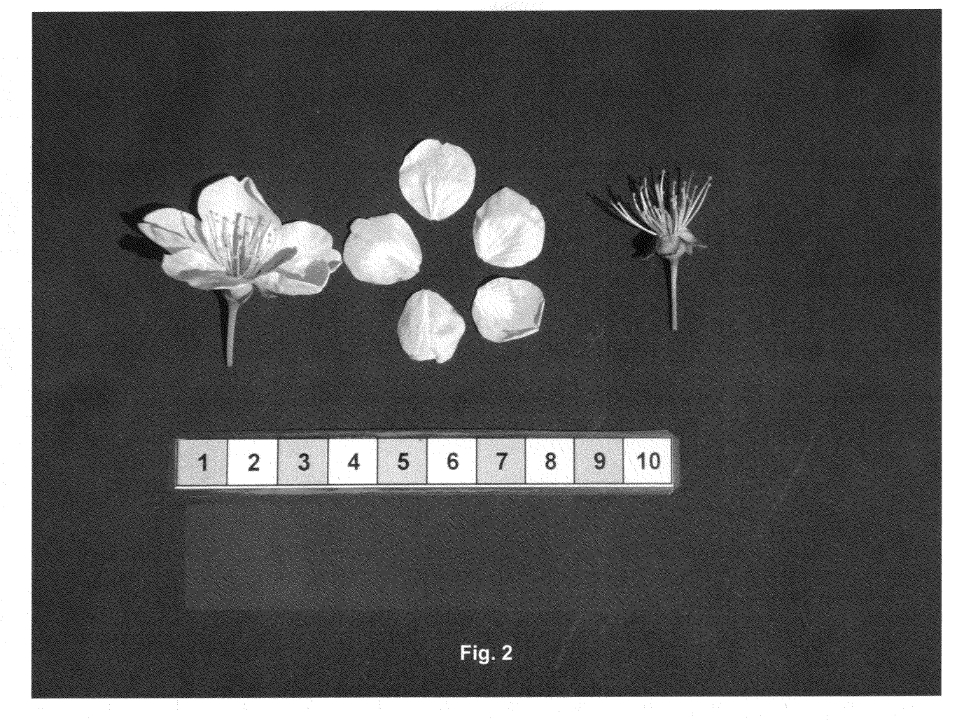

FIG. 3 3 shows the leaf profile of PA2UNIBO. Two leaves have been detached and one has been placed upper leaf surface side down to show the lower leaf surface details.

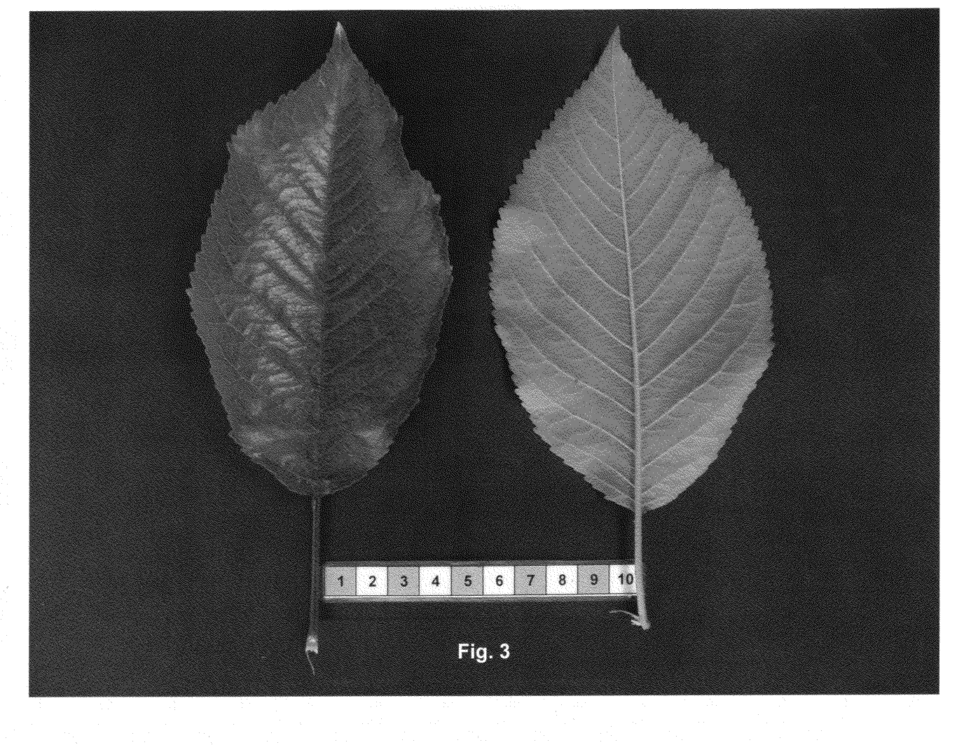

FIG. 4 shows details of fruit cropping of PA2UNIBO.

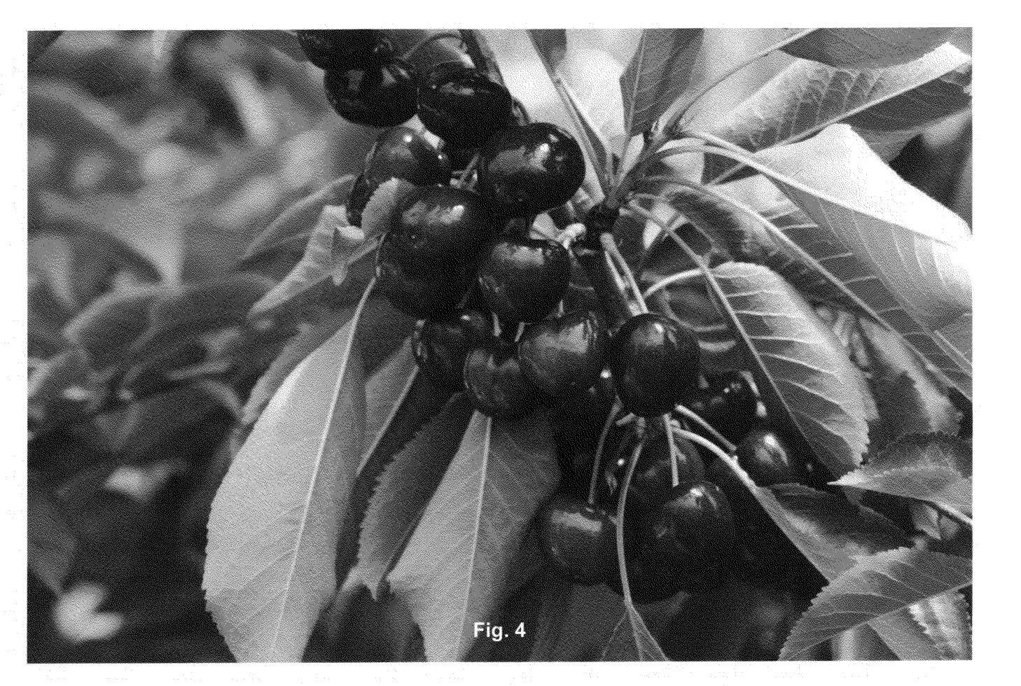

FIG. 5 shows the tree habit, with width-spread and height, of PA2UNIBO.

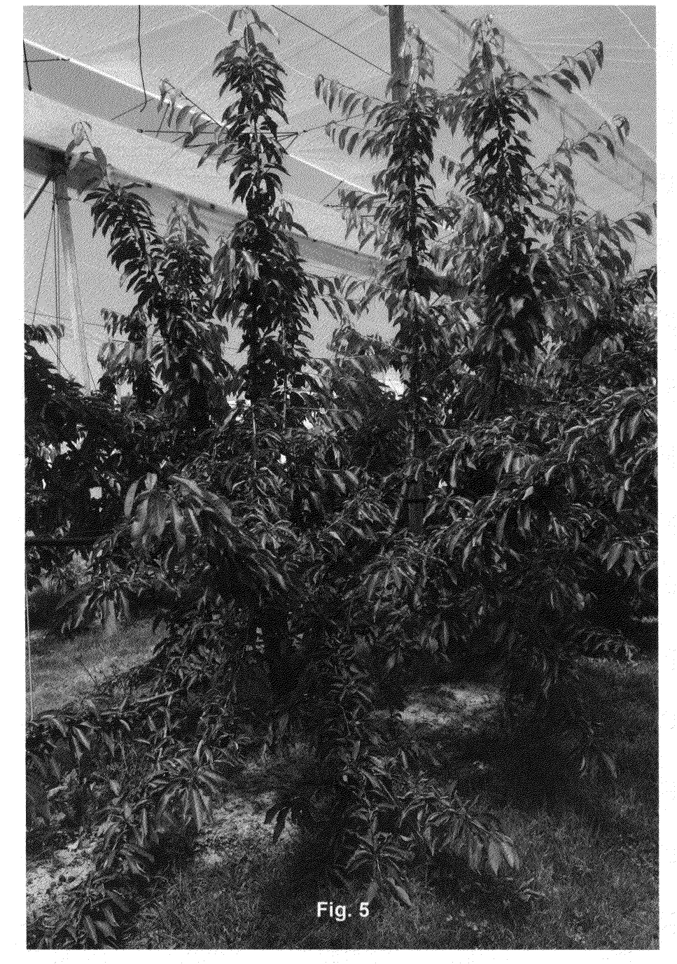

### DETAILED BOTANICAL DESCRIPTION OF THE VARIETY

### General:

Age of described tree(s).—5 years, multi-leader spindle training system.
Location of described trees(s).—Vignola, Modena Province, Italy.
Name of color chart used.—Royal Horticultural Society Colour Chart.
Name of root stock.—‘Colt’.
Tree:
   Vigor.—High.
   Height.—4-4.5 meters.
   Spread.—3 meters.
   Growth habit.—Semi-open.
   Canopy density.—Medium.
   Trunk diameter at specified height.—18-22 cm, as measured about 5 cm above graft joint.
   Trunk color.—Grayish (201A) with reddish nuances (183A).
   Bark texture.—Of average roughness.
   Tendency toward alternate bearing.—No.
   Winter hardiness.—Unknown, no winter cold damage found in this Po Valley district.
   Chilling requirement.—Unknown.
Branch:
   Length.—300-400 cm.
   Diameter.—5-6 cm.
   Crotch angle.—50°- 60° depending on training system.
   Bark color.—Reddish-brown (200C) with widespread gray overtone (201A).
   Bark texture.—Of medium fineness.
Current year shoot:
   Length.—35-50 cm.
   Color.—Reddish-brown (200C/200D) with widespread gray overtone (201A).
Winter hardiness:
   USDA hardiness zone.—Unknown.
   Chilling requirement.—Unknown.
Flower buds:
   Number per spur.—5-6.
   Shape.—Sub-globose.
   Length.—About 1 cm.
   Diameter.—Under 1 cm.
   Color.—Reddish perules (187C).
Flowers:
   Number per cluster.—Minimum 6-maximum 9 per spur; 2-3 per bud. Abundant bloom.
   Relative position of petals.—slightly overlapping petals.
   Diameter.—35-42 mm.
   Depth.—15-18 mm, measured as distance from receptacle to maximum petal aperture.
   Is pollen produced?.—Yes.
   Coloration of pollen.—Yellow (13A).
Sepal:
   Length.—6-8 mm.
   Shape.—Conical.
   Width.—5-6 mm.
   Apex.—Pointed.
   Base.—Pointed.
   Margin descriptors.—Regular margin.
   Color — upper surface.—Pale green (138C).
   Color — lower surface.—Pale green (138C).
Petals:
   Number per flower.—5.
   Shape.—Circular.
   Length.—16-20 mm.
   Width.—16-20 mm.
   Apex.—Slightly sunken.
   Base.—Slightly pointed.
   Margin.—Slightly undulated.
   Color — upper surface.—White (155B).
   Color — lower surface.—White (155B).
Bloom:
   Date of bud burst.—7-8 Apr. 2013.
   Date of first bloom.—13 Apr. 2013.
   Date of full bloom.—16 Apr. 2013.
Pedicel:
   Length.—33-36 mm.
   Diameter.—About 1.1 mm.
   Color.—Green (143B).
Pistil:
   Quantity.—1.
   Size.—18-21 mm.
   Color.—Green (143C).
Anthers:
   Quantity.—24-28: about 70% are 10-13 mm long, positioned slightly under the top of the stigma, and 1-2 anthers of many flowers are longer than the top of the stigma. The remaining 30% are 5-8 mm long. White filament (155D).
   Size.—About 1 mm in diameter.
   Color.—Yellow (15C).
Stigma:
   Quantity.—1.
   Size.—1.2-1.3 mm in diameter.
   Color.—Green (142A).
Styles:
   Quantity.—1.
   Size.—14-15 mm long.
   Color.—Pale green (142B) turning to rosè (36C) at end of bloom.
Ovary/ies:
   Quantity.—1.
   Size.—4-5 mm.
   Color.—Green (143C).
Leaves:
   Length.—14.1-15.2 cm.
   Width.—7.8-8.8 cm.
   Shape.—Elliptical.
   Apex shape.—Pointed.
   Base shape.—Pointed.
   Margin.—Fine-toothed.
   Color — upper surface.— Dark green (137A).
   Color — lower surface.— Pale green (138A/138B) with green-yellow central vein (152A/152B).
Petiole:
   Length.—4-4.8 cm.
   Diameter.—About 2 mm.
   Color.—Green-yellow (152A/152B) under and reddish upper (183A).
Fruit:
   Shape.—Heart-shaped.
   Diameter.—Mostly 30 mm.
   Height.—24-27 mm.
   Weight.—12-14 g, average weight.
   Number per cluster.—5-8 per spur.
   Skin color (include ground color and over color).—Shiny dark red (187B) tending oward blackish dark red (187A) at advanced maturity.
   Lenticels present? Density?.—Yes, medium.
   Skin thickness.—Medium.
   Skin texture.—Medium.
   Anthocyanin coloration present near skin?.—No.
   Tenacity of flesh to stone.—Low.
   Flesh color.—Rosè (52A).
   Flesh texture.—Fine.
   Juiciness.—High.
   Juice analysis (brix, acidity, tss, etc.).—18.2° Brix and 8.3 g/l malic acid (2012 data).
   Stone size.—Medium-small.
   Stone shape.—Elliptical.
   Stone color.—Pale brown (165D).
Fruit stem:
   Length.—35-38 mm.
   Diameter.—Over 1 mm.
   Color.—Green (143B).
Harvest:
   Maturity date range.—10 days after ‘Burlat’ (or about 8 days before ‘Bing’) at the same date as ‘Celeste’®Sumpaca*.
   Harvest date range (if different).—Same as above.

Because of the outstanding and unique horticultural characters of the claimed plant, especially its long harvest period and large size, this cherry can be planted to extend the cultivar's season. Although this new variety of cherry possesses the above-described characteristics, it is to be understood that variations and fluctuations may occur in the magnitude and qualities due to changes in growing and climate conditions, irrigation, fertilization, pruning and pest control.

What is claimed is:

1. A new and distinct cherry tree as herein described and illustrated.
